(19) United States
(12) Patent Application Publication
(10) Pub. No.: US 2025/0064734 A1
(43) Pub. Date: Feb. 27, 2025

(54) WATER-SOLUBLE CANNABINOID/RETINOID BLEND FORMULATIONS AND METHODS OF THEIR MAKING

(71) Applicant: SOLUSCIENCE, LLC, Lafayette, CO (US)

(72) Inventors: Thomas BLAKELEY, Boulder, CO (US); Jan ZYGMUNT, Longmont, CO (US); Basilio GARCIA, Boulder, CO (US); Jesse G. BROWN, Thornton, CO (US)

(21) Appl. No.: 18/943,673

(22) Filed: Nov. 11, 2024

Related U.S. Application Data
(63) Continuation-in-part of application No. PCT/US2023/021554, filed on May 9, 2023.
(60) Provisional application No. 63/339,899, filed on May 9, 2022.

(51) Int. Cl. A61K 9/107; A61K 8/02; A61K 8/34; A61K 8/67; A61K 31/00; A61K 31/203; A61K 45/06; A61K 47/10; A61K 47/12; A61Q 19/08

(52) U.S. Cl. CPC A61K 9/1075; A61K 8/0291; A61K 8/347; A61K 8/671; A61K 31/203; A61K 31/658; A61K 45/06; A61K 47/10; A61K 47/12; A61Q 19/08

(57) ABSTRACT

A composition containing a highly bioactive blend of cannabinoid with retinyl palmitate (or other retinyl derivative) in a form of dispersed nanoparticles in a solid biocompatible polymeric matrix. The resulting dispersion may be applied topically or administered orally in solid or liquid form to a subject to prevent and/or treat certain disorders.

### CROSS-REFERENCE TO RELATED APPLICATIONS

This application is a Continuation-in-part (CIP) of PCT application No. PCT/US2023/021554, filed May 9, 2023, which claims benefit of priority to U.S. Provisional Patent Application No. 63/339,899 filed on May 9, 2022. All of the above-mentioned applications are incorporated herein by reference in their entirety.

### FIELD OF THE INVENTION

The present invention relates to a water-soluble cannabinoid/retinoid blend formulation with enhanced bioavailability.

### BACKGROUND

Cannabinoids are hydrophobic molecules practically insoluble in water. As a result of this insolubility, the bioavailability of cannabinoids is limited and their application in medicine is a major challenge, particularly if the treatment requires relatively high dosage.

A limited number of cannabinoid formulations are currently available on the market. To overcome insolubility challenge of cannabinoids vegetable oils, alcohol and propylene glycol are used as dissolving media. But these cannabinoid solutions have limited bioavailability and stability and often cause gastrointestinal side effects. For example, Epidiolex, which is delivered orally contains cannabidiol dissolved in sesame oil and up to 10% v/v of ethanol. Marinol, which is delivered in form of capsules, includes a solution of synthetic tetrahydrocannabinol in sesame oil. Sativex, which is delivered as a liquid by spray, includes solution of an equal combination tetrahydrocannabinol and cannabidiol in ethanol and propylene glycol.

Several alternative cannabinoid formulations have been described in the patent literature. U.S. Pat. No. 10,265,293 discloses an aqueous-based oral formulation of synthetic tetrahydrocannabinol comprising propylene glycol, polyethylene glycol ethanol and water.

WO 2019135077 discloses oral formulation for a combination of two cannabinoids, which compromises an organic solvent such as propylene glycol, propylene glycol diacetate, triacetin present from 20 to 50% and hydrophilic surfactants such as poloxamers.

U.S. Pat. Nos. 10,568,865, 10,722,490, 10,842,773 and US20200121637 describe water-soluble formulation, wherein cannabinoid, Vitamin E TPGS and water were mixed and then sonicated. The liquid formulation was transformed to solid after removal of water under low pressure. Vitamin E TPGS excipient and sonication there are factors, which can significantly contribute to the high cost of the process if it is scaled up. Water removal to get solid form of the formulation is another high cost step.

US 20200222360 discloses water-soluble formulation of cannabinoids formed from tocopherol (Vitamin E), poloxamer, glycerol, carboxylic acid and water.

WO 2012/033478 discloses water-soluble formulation of cannabinoids based on Self Emulsifying Drug Delivery Systems (SEDDS). This formulation compromises glyceride-based oil, which dissolves cannabinoids, one or more lipid-based surfactants, which promote self-emulsification and co-solvent enhancing stability of an emulsion. When in contact with gastric fluids, this system emulsifies because of the presence of surfactants. Most surfactants, co-surfactants and all oil carriers included in the self-emulsifying formulations are lipid-based excipients. They lose part of their emulsification capability in gastro-intestinal track because of interaction with lipase enzymes what results in decrease of bioavailability of applied cannabinoids. In addition, the high concentrations of surfactants and oil-based carriers, which is required to keep the formulation stable, causes several gastrointestinal unwanted side effects. Presence of water miscible co-solvents, which is also needed to help in stability of emulsions, is a challenge as well because these excipients can be toxic to the human body. To properly homogenize self-emulsifying formulations expensive high shear mixers are needed what significantly can increase the cost of production on bigger scale.

U.S. Pat. No. 10,596,124 discloses lipid-based formulation for cannabinoids compromising liquid lipid like medium chain triglycerides, soya lecithin, polysorbate 80, which dissolve cannabinoid and phospholipid such as phosphatidylcholine that encapsulate the lipid solution droplets.

US 20200,170944 describes water-soluble emulsifying composition comprising cannabinoid, carrier oil based on glycerin and fatty acid monoester and emulsifier, which is sucrose fatty acid monoester or lecithin.

US 20190015383 describes water-soluble emulsifying composition including cannabinoid, carrier oil, water and polymeric carbohydrates such as starch, maltodextrin or sugar alcohol.

U.S. Pat. No. 10,709,747 discloses formulation, which uses emulsification process to encapsulate cannabinoids and includes blend of two or more surfactants where at least one surfactant is an oil carrier and the other form self-assembling emulsion, and hydrophilic co-solvent including alcohols, glycols and others.

US 20160184258 describes self-emulsifying formulation of cannabis extract comprises glyceride-based or free fatty acids oil, one or more lipid-based surfactants and co-solvent ethanol.

US 20160213624 describes formulations of hemp oil by emulsification with a surfactant/emulsifier Polysorbate 80.

WO 2015/184127 discloses formulation of synthetic tetrahydrocannabinol, Drobinol using vegetable oils and different glycerides and ethanol; the patent reveals also formulation of Drobinol based on a mix of polyethylene glycol and propylene glycol and optionally water.

US 20190167583 described oral cannabinoid formulation including a lipid solvent, a sweetener and ethanol.

US 20200297690 describes composition of water-soluble powder comprising cannabinoid, phospholipids or glycerides, sugar alcohols such as xylitol or mannitol, polysaccharide maltodextrin and amino acids.

US 20190365667 describes an oral cannabinoid formulation comprising one or more cannabinoids dissolved in a solvent system consisting of water and non-ionic surfactant such as Polysorbate 80, Kolliphor® RH40, polyoxyethylene or poloxamers. Preferred non-ionic surfactant is Kremophor® RH 40, which is derivative of hydrogenated castor oil and ethylene oxide.

US 20190030170 describes a water-soluble cannabinoid formulation based on inclusion complex of cannabinoid with sulfobuty ether of beta cyclodextrin, Captisol. However, Captisol is synthetic high-cost cyclodextrin and the scale-up production of the complex may not be economically viable.

One of the objectives of the present invention is to increase bioavailability of the blend of retinol derivative and cannabinoid and improve transdermal delivery of the blend in topical application and oral delivery in solid dosage application.

### SUMMARY

This disclosure provides a composition and formulation thereof. In one embodiment, the composition contains a bioactive formulation of retinyl palmitate blended with cannabinoid. In another embodiment, the disclosed formulation forms a solid uniform composite. In another embodiment, the composite is a homogenic mixture of (1) cannabinoid, (2) retinyl palmitate and (3) a natural stabilizer, dispersed as nanoparticles in a polymeric hydrophilic matrix. The amount of retinyl palmitate is about 0.5%-10% by weight, cannabinoid is about 0.01%-20% by weight, and natural stabilizer is about 0.5%-10% by weight. Examples of natural stabilizer include but are not limited to ascorbic acid, citric acid, ascorbyl palmitate, alpha-tocopherol, butylated hydroxytoluene, butylated hydroxyanisole, propyl gallate, sodium sulphite, sodium metabisulphite, sodium formaldehyde sulphoxylate and mixtures thereof. In one aspect, the polymeric hydrophilic matrix is biocompatible. In another aspect, the nanoparticles have a median diameter of between 10 nm to 50 nm.

In one embodiment, a process is disclosed for making the composition, which includes melting and effective stirring of a mixture of retinyl palmitate, a cannabinoid, a polymeric hydrophilic matrix to obtain homogenic liquid melt via thermal fusion. In one aspect, thermal fusion is performed by melting components together and mix to disperse one component in another one. In one aspect, a natural stabilizer is added to the composition to help stabilize the composition. Examples of polymeric hydrophilic matrix include but are not limited to poloxamer, polyvinylpyrrolidone, polyvinylpyrrolidone-polyvinylacetate copolymer, polyvinyl acetate phthalate, polyvinyl alcohol, polycaprolactam, polylactic acid, polyglycolic acid, polymethacrylic polymers, Soluplus, Poloxamers, polyvinyl acetate polyethylene glycol graft co-polymers (e.g., Soluplus.RTM.), hydroxypropyl methyl cellulose phthalate, cellulose acetate phthalate, cellulose acetate trimellitate, cellulose acetate phthalate, methyl cellulose acetate phthalate, hydroxyethyl cellulose, hydroxypropyl cellulose, hydroxypropyl methylcellulose, hydroxypropyl cellulose acetate phthalate, cellulose acetate terephthalate, cellulose acetate isophthalate, hydroxypropyl methyl cellulose acetate succinate, hydroxypropyl methyl cellulose propionate succinate, carboxymethyl ethylcellulose, sodium carboxymethylcellulose, and mixtures thereof. In another aspect, no organic solvent is used. In another aspect, the melt is cooled to yield a solid composite containing highly dispersed cannabinoid/retinyl palmitate blend in the polymeric matrix.

In another embodiment, the disclosed composite can be used in topical application in a form of gel, cream or ointment. In another embodiment, the disclosed composite can also be used in oral application as a solid dose in a form of a tablet or a capsule.

In one aspect, the disclosed composition can be used to treat acne, cancer and other disorders. The composition can be applied topically to a subject or taken orally. For example, when the composition is applied topically to a subject to treat acne, the bioactive blend is delivered via transdermal process into the blood circulation system of the subject.

In another aspect, when the composition is taken orally, for example to treat cancer, the solid dose of the blend is dissolved in gastrointestinal fluids and forms a bioactive micellar dispersion. The micellar dispersion is conveyed to the intestine where it is absorbed via the intestinal wall and transported to the liver where retinol undergoes nominal changes through metabolism and then is distributed into the blood circulation system.

In one embodiment, the formulation of the composition for topical application includes one or more anti-ageing ingredients (agents) The amount of the one or more anti-ageing ingredients is from 0.1%- 10%, or 0.1%-5%, or 0.2%- 4%, or 0.3%- 3% by weight. By way of example, the anti-ageing ingredients may include but are not limited to peptlium, or hyaluronic acid.

In one aspect, the anti-ageing ingredients may be water-soluble. Examples of the water-soluble anti-ageing ingredients include but are not limited to azelaic acid, niacinamide, lysine, water-soluble oligopeptides, collagen, and green tea extract.

In another embodiment, the formulation of the composition may contain one or more cosmetic preservatives, for example Germall plus, which contains diazolidinyl urea and Iodopropynyl butylcarbamate. Other examples of water-soluble cosmetic preservatives include but are not limited to: salts of formic acid, citric acid, sorbic acid, salicylic acid and dehydroacetic acid; phenoxyethanol, ethylhexylglycerin, benzyl alcohol, grapefruit seed extract, rosemary extract.

According to the present disclosure, a water-soluble solid dispersion formulation is provided. In one embodiment, the formulation comprises one or more cannabinoids and a retinyl derivative dispersed in a pharmaceutically acceptable hydrophilic polymeric matrix.

In another embodiment, the solid dispersion formulation according to the present disclosure may contain a mixture of cannabinoids.

In another embodiment, the solid dispersion formulation can be applied in solid, liquid or gel form and delivered orally, sublingually or transdermally.

In one aspect, the polymeric matrix may be selected from the group of amphiphilic polymers such as poloxamers. In another aspect, the polymeric matrix is Poloxamer 407. Poloxamer 407 is tri-block polymer built from polyoxyethylene and polyoxypropylene blocks. It is soluble in water and approved by FDA to use in humans. In another aspect, the hydrophilic polymeric matrix is present from 60% to 99%, or 70% to 95%, or 70% to 90% by weight of the solid dispersion formulation.

In another embodiment, the cannabinoid material is present from 0.01% to 20%, or from 0.5% to 10% by weight of the solid dispersion formulation. In one aspect, the one or more cannabinoids are phytocannabinoids or synthetic cannabinoids. The cannabinoid material can be in the form of a liquid obtained by extraction from plant and purified by vacuum distillation or in the form of solid isolate purified by chromatography and crystallization. In another aspect, examples of the one or more cannabinoids included in the cannabinoid material may include but are not limited to: cannabichromene (CBC), cannabichromenic acid (CBCV), cannabidiol (CBD), cannabidiolic acid (CBDA), cannabidivarin (CBDV), cannabigerol (CBG), cannabigerol propyl variant (CBGV), cannabicyclol (CBL), cannabinol (CBN), cannabinol propyl variant (CBNV), cannabitriol (CBO), tetrahydrocannabinol (THC), tetrahydrocannabinolic acid (THCA), tetrahydrocannabivarin (THCV) or tetrahydrocannabivarinic acid (THCVA). In another aspect, the one or more cannabinoids are cannabidiol (CBD), cannabinol (CBN) or cannabigerol (CBG).

In a further embodiment, the solid dispersion formulation may be mixed with cosmetic components designed to moisturize skin and help penetration of formulated cannabinoid into or through the skin and is in the form of a serum, an ointment, a cream, an emulsion, a lotion, a paste, an unguent, a gel or a sunscreen. The solid dispersion may include the cannabinoid material mixed with the retinol-based material or it may include the retinol-based material only.

In one aspect, the solid dispersion formulation can be used in a solid form of capsules, tablets and other solid drug delivery methods.

### DETAILED DESCRIPTION

### Dermatological test

The obtained solid dispersion of cannabidiol/retinyl palmitate blends was preliminary tested in form of cosmetic formulations by volunteers to determine if they are safe and effective. The tested samples showed the anticipated results such as reduction or elimination of acne, elimination or reduction forehead lines, bunny lines crows feet, gummy smiles, lip lines dimpled chin, bags under eyes, black spots, reducing wrinkles, shrink pores size, improve skin texture and tone and no safety concerns were noticed.

These data are significant because they showed for the first time that such cannabinoid/retinol blends of the present invention can improve significantly the biological effectiveness of the components in a positive dermatologic outcome and at the same time provides stable and palatable product.

### Preservatives

Microorganisms develop readily in an aqueous environment and components of cosmetic formulations such as matrixes, nutrients, and vitamins are excellent food for theirs grow.

To protect the disclosed composition in the present invention, solid or liquid cosmetic formulations or preservatives against grow of microorganisms, mold, and fungus and to maintain their microbiological quality the presence of specific preservatives may be added.

The most common cosmetic preservatives are organic acids such as benzoic acid and its derivatives Parabens (methylparaben, ethylparaben, propylparaben, butylparaben, isobutylparaben), sorbic acid, levulinic acid, and anisic acid. They are also formaldehyde, glycol ethers such as phenoxyethanol and caprylyl glycol, imadozolidinyl and diazolidinyl ureas and Isothiazolinones such as methylisothiazolinone.

In one embodiment, the formulation of the composition may contain one or more cosmetic preservatives, for example Germall plus, which contains diazolidinyl urea and Iodopropynyl butylcarbamate.

Other examples of water-soluble cosmetic preservatives include but are not limited to: salts of formic acid, citric acid, sorbic acid, salicylic acid and dehydroacetic acid; phenoxyethanol, ethylhexylglycerin, benzyl alcohol, grapefruit seed extract, rosemary extract.

The present disclosure also includes the following embodiments.

Item 1. A composition comprising a cannabinoid material, a retinyl derivative, and a polymeric hydrophilic matrix, wherein said cannabinoid material and said retinyl derivative are dispersed as nanoparticles in said polymeric hydrophilic matrix.
Item 2. The composition of Item 1, wherein the cannabinoid is present in said composition at 0.01%-20% by weight, retinyl derivative is present in said composition at 0.5%-10% by weight, and polymeric hydrophilic matrix is present in said composition from 60% to 99% by weight.
Item 3. The composition of any preceding items, wherein said polymeric hydrophilic matrix is poloxamer.
Item 4. The composition of any preceding items, further comprising a natural stabilizer.
Item 5. The composition of Item 4, wherein the natural stabilizer is ascorbic acid.
Item 6. The composition of any preceding items, wherein the composition is in a form selected from the group consisting of a solid form, a liquid form and a micellar dispersion form.
Item 7. The composition of any preceding items, wherein the composition is in a solid form and is soluble in water.
Item 8. The composition of any preceding items, wherein the retinyl derivative is retinyl palmitate.
Item 9. The composition of any preceding items, wherein the cannabinoid material comprises one or more cannabinoids, and the cannabinoid being selected from the group consisting of tetrahydrocannabinol, cannabidiol, cannabigerol, cannabigerolovarin, cannabichromene, cannabidivarin, tetrahydrocannabivarin, cannabivarin, cannabinol, and isomers thereof, and mixtures thereof.
Item 10. A composition comprising a retinyl derivative, and a polymeric hydrophilic matrix, wherein said retinyl derivative is dispersed as nanoparticles in said polymeric hydrophilic matrix.
Item 11. A pharmaceutical composition comprising the composition of any preceding items, wherein the composition of any preceding items is dissolved in water to form a colloidal dispersion.
Item 12. The pharmaceutical composition of Item 11, further comprising a cosmetic preservative.
Item 13. The pharmaceutical composition of Item 12, wherein a cosmetic preservative is Germall plus, said Germall plus comprising diazolidinyl urea and Iodopropynyl butylcarbamate.
Item 14. The pharmaceutical composition of any of Items 11-13, further comprising an anti ageing agent.
Item 15. The pharmaceutical composition of Item 14, wherein the anti-ageing agent is selected from the group consisting of peptlium, and hyaluronic acid.
Item 16. A method of treating a disorder in a subject, comprising administer the composition of claim 11 to the subject.
Item 17. The method of Item 16, wherein the disorder is a skin disease.
Item 18. A method of making a pharmaceutical composition, comprising
a) mixing a cannabinoid material, a retinyl derivative, and a polymeric hydrophilic matrix to form a solid dispersion,
b) dissolving the solid dispersion from step (a) in water to form a colloidal dispersion (or micellar dispersion), and
3) obtain the colloidal dispersion as the pharmaceutical composition.

The disclosure will now be illustrated with working examples, and which is intended to illustrate the working of disclosure and not intended to restrictively any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

### EXAMPLES

### Example 1 Preparation of retinyl palmitate/cannabinoid Blend Flakes

A mixing vessel was charged with 88.0 g of solid Poloxamer and the vessel was placed into the preheating to 90° C. oil bath. The solid was mixed by mechanical stirrer at 50 RPM until it melted into a creamy liquid paste and then 2.0 g of ascorbic acid was added and mixed for an additional 10 minutes. Next 5.0 g of retinyl palmitate and 5.0 g of cannabinoid were added stepwise and when the full homogeneity was achieved the mixer RPM was increased to 200 and the resulting mixture was stirred for 2 hours at 90° C. Then the melt was transferred to a tray and the tray was well sealed and placed in a freezer at −20° C. overnight. The resulting cold slabs were broken to smaller pieces and grinded in a motorized grinder to make flakes. The yield was 95%.

### Example 2 Preparation of Micellar Solution of retinyl palmitate/cannabinoid Blend (Version 1)

Examples 2 and 3 show two different versions of protocols for preparing the micellar solution of retinyl palmitate/cannabinoid blend. The mixing vessel was charged with 88.0 g of solid Poloxamer and the vessel was placed into the preheating to 90° C. oil bath. The solid was mixed by mechanical stirrer at 50 RPM until it melted into a creamy liquid paste and then 2.0 g of ascorbic acid was added and mixing was continued for 10 minutes. Next 5.0 g of retinyl palmitate and 5.0 g of cannabinoid were added stepwise and when the full homogeneity was achieved the mixer RPM was increased to 200 and the resulting mixture was stirred for 2 hours at 90° C. Then the oil bath was removed and at continuous stirring 875 mL of distilled water was added slowly and after that 5.0 g of natural preservative Germall plus and 20.0 g of natural anti-ageing ingredient Peptilium were added. The mixture was stirred overnight and the resulting fully homogenized liquid product was released into the appropriate packaging or storage container.

### Example 3 Preparation of Micellar Solution of retinyl palmitate/cannabinoid Blend (Version 2)

The mixing vessel was charged with 88.0 g of solid Poloxamer and the vessel was placed into the preheating to 90° C. oil bath. The solid was mixed by mechanical stirrer at 50 RPM until it melted into a creamy liquid paste and then 2.0 g of ascorbic acid was added and mixing was continued for 10 minutes. Next 5.0 g of retinyl palmitate and 5.0 g of cannabinoid were added stepwise and when the full homogeneity was achieved the mixer RPM was increased to 200 and the resulting mixture was stirred for 2 hours at 90° C. Then the oil bath was removed and at continuous stirring 865 mL of distilled water was added slowly and after that 5.0 g of natural preservative Germall plus and 20.0 g of natural anti-ageing ingredient Peptilium were added. The mixture was stirred overnight and to the resulting fully homogenized liquid was added 10.0 g of hyaluronic acid and stirring was continued for 30 minutes. The final liquid product was released into the appropriate packaging or storage container.

### Example 4. Improved Preparation of Micellar Solution of cannabidiol—SoluRetinol Daytime Variation. Scale-Up Production Process (6 L Batch)

To the process vessel that had been preheated to 90° C., 480 g of Poloxamer and 12 g of Ascorbic Acid were added and mixed at a low RPM, gradually increasing the speed until a pasty melt forms. Once the melt became homogenic, 60 g of CBD isolate was added and the RPM was increased to achieve thorough agitation without causing excessive splatter inside the vessel. The melt was continuously agitated for 1 hour. In a separate container 5219.4 g of water, 30 g of Germall Plus, 90 g of Optiphen, and 108 g of Ascorbic Acid were combined and stirred until a homogeneous mixture was achieved. The heat in the process vessel was turned off and the resulting mixture was slowly added to it and the RPM is increased to a level just below where a vortex would form. Once the mixture has reached full homogenization, the final liquid product was released into the appropriate packaging or storage containers. Recovery yield: 95%.

### Example 5. Improved preparation of Micellar Solution of retinyl palmitate/cannabidiol Blend—SoluRetinol Nighttime Variation. Scale-Up Production Process (6 L Batch)

To the process vessel that had been preheated to 90° C., 480 g of Poloxamer and 12 g of Ascorbic Acid were added and mixed at a low RPM, gradually increasing the speed until a pasty melt forms. Once the melt became homogenic, 60 g of CBD isolate and 30 g retinyl palmitate were added and the RPM was increased to achieve thorough agitation without causing excessive splatter inside the vessel. The melt was continuously agitated for 1 hour. In a separate container 5237.4 g of water, 30 g of Germall Plus, and 90 g of Optiphen, were combined and stirred until a homogeneous mixture was achieved. The heat in the process vessel was turned off and the resulting mixture was slowly added to it and the RPM is increased to a level just below where a vortex would form. Once the mixture has reached full homogenization, 60 g of hyaluronic acid was added and after the full homogenization was achieved again, the final liquid product was released into the appropriate packaging or storage containers. Recovery yield: 95%.

### Example 6. Preparation of retinyl palmitate/salicylic acid Blend Liquid Concentrate (SoluClear); Scale-Up Production Process (3L Batch)

To the process vessel that had been preheated to 90° C., 213.89 g of Poloxamer and 6 g of Ascorbic Acid were added and mixed at a low RPM, gradually increasing the speed until a pasty melt forms. Once the melt became homogenic, 60.90 g of CBD isolate and 1.50 g of peppermint oil were added and the RPM was increased to achieve thorough agitation without causing excessive splatter inside the vessel. The melt was continuously agitated for 1 hour. In a separate container 2640 g of water, 15 g of Germall Plus, and 45 g of Optiphen, are combined and stirred until a homogeneous mixture was achieved. The heat in the process vessel was turned off and the resulting mixture was slowly added to it and the RPM is increased to a level just below where a vortex would form. Once the mixture had reached full homogenization 18 g of salicylic acid was added, and agitation was continued. Once the solution has reached full homogenization, the final liquid product was released into the appropriate packaging or storage containers. Recovery yield: 95%.

### Example 7 Use of SoluRetinol

SoluRetinol is a liquid composition that contains poloxamer, retinol (in form of palmitate prodrug) and CBD. Methods of preparing SoluRetinol are described in Examples 4 and 5. The products prepared according to Examples 4 and 5 work in tandem to improve skin appearance; the product from Example 4 is applied during daytime (SoluRetinol daytime) and the product from Example 5 is applied at nightime (SoluRetinol nighttime). The SoluRetinol application is an anti-aging treatment and improves skin conditions. It is also suitable for mature and time-scarred skin.

When SoluRetinol is applied to the skin at a dosage of 1.5-3%, or 2-2.5%, or 2.2-2.5% (w/w) of retinol and 1.5-3% or 2-2.5%, or 2.2-2.5% (w/w) of CBD per single use, it initiates several beneficial changes at the cellular and structural levels of the skin. In some embodiments, the SoluRetinol at the above dosage is applied to skin for a course of 3-90 days, or at least 7 days, or at least 20 days for effects to be visible by eyes.

Enhanced Penetration and Controlled Delivery by poloxamer:

Poloxamer acts as a thermosensitive hydrogel, improving the bioavailability, delivery and stability of active ingredients like retinol and CBD. Upon application to the skin, poloxamer-based formulation adapts to skin temperature, enhancing ingredient penetration into the skin's epidermal and dermal layers, where they can effectively target cellular structures.

Collagen Synthesis and Anti-Aging Effects by retinol: Retinol, the alcohol form of vitamin A and its ester prodrugs, stimulate fibroblasts in the dermis to produce collagen and elastin. This process improves skin elasticity and firmness, addressing signs of aging such as fine lines and wrinkles. Retinol also accelerates cell turnover, promoting the shedding of older, damaged skin cells and revealing fresher cells beneath. The poloxamer formulation stabilizes retinol, reducing its oxidation and maximizing its effectiveness over a prolonged period.

### Anti-Inflammatory and Antioxidant Effects by CBD:

Cannabidiol (CBD) is known for its potent anti-inflammatory and antioxidant properties. Upon absorption, CBD modulates the skin's inflammatory response, helping reduce redness, irritation, and oxidative stress caused by free radicals. This anti-inflammatory action supports skin healing and enhances skin barrier function, improving resistance to environmental stressors. The combination of poloxamer with CBD enhances the bioavailability of CBD, allowing for more effective interaction with cannabinoid receptors in the skin.

### Synergistic Antioxidant and Protective Effects:

Together, retinol and CBD work synergistically to reduce oxidative stress on the skin. Retinol promotes cellular renewal and CBD modulates inflammation and neutralizes free radicals. The combination of these ingredients helps to repair, protect, and rejuvenate the skin, resulting in improved texture, reduced wrinkle depth, and enhanced radiance with a synergistic effect that is more than 20%, or more than 40%, or more than 60% greater than the additive effects if the retinol and CBD are applied separately. Some or all these effects may be quantified by professional eyes or by measurement using specialized instruments.

### Non-Irritating, PH-Balanced Delivery System:

Poloxamer hydrogel formulation stabilizes the serum and reduces the likelihood of irritation from retinol by controlling its release. This gradual release mechanism allows for a sustained effect of active ingredients while minimizing potential irritation typically associated with retinol-based products, making it suitable for sensitive skin types.

In summary, applying a poloxamer-based liquid composition containing retinol and CBD leads to a multi-targeted approach to skin care. It enhances the skin's structural integrity, increases hydration, modulates inflammatory responses, and promotes cellular renewal, ultimately improving skin texture, reducing signs of aging, and strengthening the skin barrier.

### Example 8 Preparation and Use of SoluClear

SoluClear (also referred to as SoluAcne) is a topical skin treatment formulation containing poloxamer, cannabidiol and salicylic acid, a known anti-acne agent. This formulation offers multiple therapeutic advantages due to enhanced solubility, improved penetration, and a more controlled release profile. SoluClear regulates wrinkles and atrophy in the human skin comprising chronic treatment of the skin with a safe and effective amount of CBD and salicylic acid.

Enhanced Skin Penetration by poloxamer:

Poloxamer, a nonionic surfactant, acts as a solubilizer and enhances the permeation of active ingredients through the skin. It forms micelles that improve the solubility of hydrophobic agents like CBD and facilitate the transport of salicylic acid, which is otherwise limited by its poor solubility in water. By increasing the delivery of these agents into the deeper layers of the skin, poloxamer-based formulations target sebaceous glands more effectively, where acne forms due to inflammation, microbial colonization, and sebum buildup.

### Anti-Inflammatory Action (CBD):

CBD's anti-inflammatory properties modulate acne-related inflammation by reducing cytokine production, particularly interleukin-1 (IL-1) and tumor necrosis factor-alpha (TNF-α). It also inhibits lipid production in sebocytes, reducing the excess sebum that can lead to acne lesions. Additionally, CBD's antioxidative properties help in neutralizing free radicals, preventing damage to skin cells that exacerbate acne inflammation. These actions are beneficial in treating inflammatory acne, as CBD not only reduces inflammation but also soothes irritation associated with existing acne lesions.

Comedolytic Effect of (salicylic acid):

Salicylic acid is known for its keratolytic and comedolytic properties, effectively exfoliating dead skin cells and unclogging pores. It penetrates the follicular canal due to its lipid solubility, breaking down desmosomes (the connections between keratinocytes) and facilitating desquamation. This mechanism prevents the formation of comedones, a core component of acne development, by reducing cellular debris and excess sebum. The use of poloxamer to solubilize salicylic acid allows for a more consistent and gentle delivery, reducing the potential for irritation often seen with salicylic acid treatments.

### Synergistic Effects and Safety Profile:

By using CBD and salicylic acid in a poloxamer gel, the formulation benefits from the anti-inflammatory properties of CBD and the exfoliating, comedolytic action of salicylic acid, resulting in a treatment that targets multiple pathogenic factors of acne.

The slow-release characteristic of poloxamer also enhances the tolerability of both agents, reducing irritation, a common side effect in acne therapy. This controlled release supports skin barrier function, making the formulation suitable for patients who are sensitive to traditional acne treatments.

In summary, a CBD and salicylic acid formulation solubilized in poloxamer offers a comprehensive, multi-modal approach to acne treatment, targeting inflammation, excess sebum production, and comedogenesis while enhancing patient compliance through a more tolerable delivery system. The synergistic effect of CBD and salicylic acid on treating acne, controlling inflammation, reducing excess sebum production, and comedogenesis is more than 20%, or more than 40%, or more than 60% greater than the additive effects if the CBD and salicylic acid are applied separately. Some or all of these effects may be quantified by professional eyes or by measurement using specialized instruments.

### REFERENCES

The following references, patents or patent applications as well as all other references, patents or patent applications cited in this disclosure are incorporated herein in their entirety:
US 2022/0142969: “Water soluble cannabinoid formulations and methods of their making”; May 12, 2022; J. Zygmunt, T. Blakeley.
U.S. Pat. No. 7,244,718: “Water soluble prodrugs of hindered alcohols”; July 17, 2007; Stella, Valentino J; Zygmunt, Jan; Georg, Ingrid Gunda; Safadi, Muhammad S.
U.S. Pat. No. 6,872,838: “Water soluble prodrugs of hindered alcohols”; March 29, 2005; Stella, Valentino J; Zygmunt, Jan; Georg, Ingrid Gunda; Safadi, Muhammad S.
U.S. Pat. No. 6,451,776: “Water soluble prodrugs of hindered alcohols”; September 17, 2002; Stella, Valentino J; Zygmunt, Jan; Georg, Ingrid Gunda; Safadi, Muhammad S.
U.S. Pat. No. 6,204,257; “Water soluble prodrugs of hindered alcohols”; March 20, 2001; Stella, Valentino J; Zygmunt, Jan J.; Georg, Ingrid Gunda; Safadi, Muhammad S.
U.S. Pat. No. 5,985,856: “Water soluble prodrugs of secondary and tertiary amine containing drugs and methods of making thereof”; November 16, 1999; Stella, Valentino; Krise, Jeffrey P.; Zygmunt, Jan; Georg, Ingrid Gunda.

1. A pharmaceutical composition for treating a skin disease, comprising a cannabinoid material, a retinyl derivative, and a polymeric hydrophilic matrix, wherein said cannabinoid material and said retinyl derivative are dispersed as nanoparticles in said polymeric hydrophilic matrix, wherein said cannabinoid material, and said retinyl derivative are each present in the pharmaceutical composition at an effective amount for treating said skin disease.

2. The pharmaceutical composition of claim 1, wherein the cannabinoid is present in said composition at 0.01%-20% by weight, retinyl derivative is present in said composition at 0.5%-10% by weight, and polymeric hydrophilic matrix is present in said composition from 60% to 99% by weight.

3. The pharmaceutical composition of claim 1, wherein said polymeric hydrophilic matrix is poloxamer.

4. The pharmaceutical composition of claim 1, further comprising a natural stabilizer, ascorbic acid.

5. The pharmaceutical composition of claim 1, wherein the pharmaceutical composition is in a form selected from the group consisting of a solid form, a liquid form and a micellar dispersion form.

6. The pharmaceutical composition of claim 1, wherein the composition is in a solid form and is soluble in water.

7. The pharmaceutical composition of claim 1, wherein the retinyl derivative is retinyl palmitate.

8. The pharmaceutical composition of claim 1, wherein the cannabinoid material comprises one or more cannabinoids, and the cannabinoid being selected from the group consisting of tetrahydrocannabinol, cannabidiol, cannabigerol, cannabigerolovarin, cannabichromene, cannabidivarin, tetrahydrocannabivarin, cannabivarin, cannabinol, and isomers thereof, and mixtures thereof.

9. The pharmaceutical composition of claim 1, wherein the cannabinoid material and retinyl derivative are present in an effective amount such that the cannabinoid material and the retinyl derivative act synergistically to treat the skin disease.

10. The pharmaceutical composition of claim 1, further comprising Germall Plus and/or Optiphen.

11. A cosmetic composition for improving a skin condition, comprising a cannabinoid material, a retinyl derivative, and a polymeric hydrophilic matrix, wherein said cannabinoid material and said retinyl derivative are dispersed as nanoparticles in said polymeric hydrophilic matrix, wherein said cannabinoid material, and said retinyl derivative are each present in the cosmetic composition at an effective amount for improving the skin condition.

12. The cosmetic composition of claim 11, wherein the cannabinoid material and retinyl derivative are present in an effective amount such that the cannabinoid material and the retinyl derivative act synergistically to improve the skin condition.

13. The cosmetic composition of claim 12, further comprising a first preservative.

14. The cosmetic composition of claim 13, wherein the first preservative is Germall plus, said Germall plus comprising diazolidinyl urea and Iodopropynyl butylcarbamate.

15. The cosmetic composition of claim 13, further comprising a second preservative Optiphen.

16. A pharmaceutical composition comprising cannabidiol (CBD), salicylic acid and a polymeric hydrophilic matrix, wherein CBD is dispersed as nanoparticles in said polymeric hydrophilic matrix.

17. The pharmaceutical composition of claim 14, further comprising an anti-ageing agent.

18. The pharmaceutical composition of claim 16, further comprising Germall Plus and/or Optiphen.

19. A method of treating a skin disease in a subject, comprising administering the pharmaceutical composition of claim 1 to the subject.

20. A method of improving a skin condition in a subject, comprising administering the cosmetic composition of claim 11 to the subject.

21. A method of treating a skin disease in a subject, comprising administering the pharmaceutical composition of claim 16 to the subject.

22. A method of making the pharmaceutical composition of claim 1, comprising
a) mixing a cannabinoid material, a retinyl derivative, and a polymeric hydrophilic matrix to form a solid dispersion,
b) dissolving the solid dispersion from step (a) in water to form a colloidal dispersion (or micellar dispersion), and
3) obtain the colloidal dispersion as the pharmaceutical composition.
